(19) United States
(12) Patent Application Publication
(10) Pub. No.: US 2014/0379090 A1
(43) Pub. Date: Dec. 25, 2014

(54) IN-VIVO CONDITION MONITORING OF METALLIC IMPLANTS BY ELECTROCHEMICAL TECHNIQUES

(71) Applicants: Nikitas Diomidis, Lausanne (CH); Stefano Mischler, La Sarraz (CH); Eric Meurville, Chaffois (FR)

(72) Inventors: Nikitas Diomidis, Lausanne (CH); Stefano Mischler, La Sarraz (CH); Eric Meurville, Chaffois (FR)

(73) Assignee: ECOLE POLYTECHNIQUE FEDERALE DE LAUSANNE (EPFL), Lausanne (CH)

(21) Appl. No.: 14/237,655

(22) PCT Filed: Aug. 8, 2012
(86) PCT No.: PCT/IB2012/054046

§ 371 Date: May 29, 2014

(30) Foreign Application Priority Data
Aug. 8, 2011 (EP) 11176802.4

(51) Int. Cl. A61F 2/46; A61F 2/38; A61F 2/32

(52) U.S. Cl. CPC A61F 2/4657; A61F 2/32; A61F 2/38
USPC 623/20.17; 623/23.49; 623/22.11

(57) ABSTRACT

The invention relates to a replacement metallic prosthesis to be implanted which contains means monitor its condition during use in order to allow an early detection of failure or insufficient functionality, wherein said means comprise implanted sensors and electronics (3) and a remote device (9), to measure the implant's function and degradation during its life span, wherein said sensors are electro chemical sensors with electrodes (1). The prosthesis according to the invention may also be used to promote bone growth. The invention also relates to a method using the device of the invention.

### CROSS-REFERENCE TO RELATED APPLICATION

The present application claims the benefit of the priority of European patent application Number 11176802.4, filed on Aug. 8, 2011 in the name of ECOLE POLYTECHNIQUE FEDERALE DE LAUSANNE (EPFL), the entire disclosure of which is incorporated herein by reference.

### AIM AND FIELD OF THE INVENTION

The present invention concerns the field of metallic implants that are used in a human or animal body, such as replacement prostheses, implants etc.

More specially, the present invention relates to means that allow an in-vivo monitoring of such prostheses or implants.

Indeed out of the million hip and knee replacement prostheses that are implanted each year in the EU and US, none contains a way to monitor their condition during use, which would allow an early detection of failure or insufficient functionality.

We aim to develop a prototype system comprising an implanted sensors and electronics and a remote device, to measure the implant's function and degradation during its life span. While the overall objective is to propose systems that can be used with any metallic implant, on embodiment will initially focus on hip joint replacement prostheses.

### FUNDAMENTALS AND BACKGROUND OF THE INVENTION

Metallic materials implanted in living tissues are subject to the corrosive environment of body fluids. The materials used are selected for their high corrosion resistance in order to avoid contamination of the surrounding tissues by corrosion products such as metallic ions.

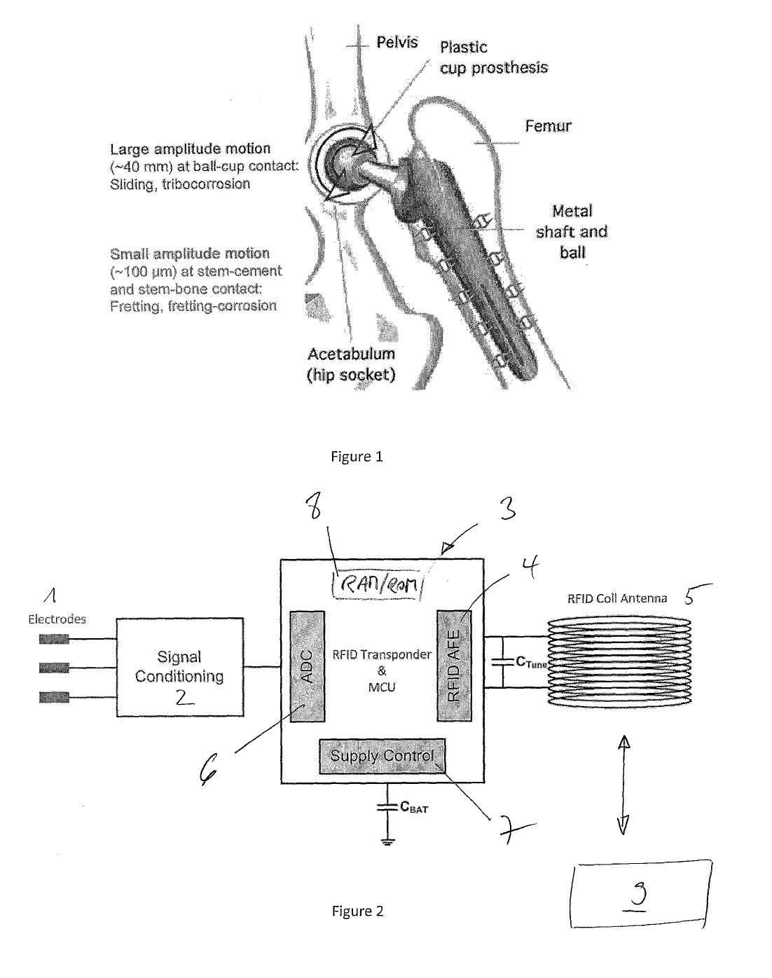

This resistance to corrosion is usually achieved by the spontaneous formation of a protective surface oxide film which shields the metal from the environment. When a mechanical interference, i.e. relative motion, takes place, the surface film may be damaged or removed and thus uncovering the base metal which is prone to corrosion (FIG. 1). Furthermore, uniform corrosion of metallic materials may take place due to inflammation of the surrounding tissues which leads to a change in pH.

The prior art includes for example the following documents.

US 2006/0047283: This document relates to an in-vivo orthopedic implant diagnostic device for sensing load, wear and infection. The sensor uses RF technology or other means to remotely transmit data. This document mentions that the sensor package may incorporate a chemical sensing element (paragraph [0038]). This sensor comprises microcantilevers coated with aptameric receptors or antibodies specific to inflammatory chemicals typically associated with infection (paragraph [0059]).

The disclosed chemical sensor is used to indicate the presence of an infection and not to measure the implant's function and degradation during its life span.

US 2005/0012610: This document concerns a joint endoprosthesis comprising a sensor adapted for monitoring the ambient conditions. The sensor may be a temperature sensor but it can be replaced by a pH sensor. Paragraph [0013] also mentions that “sensors of this type can often be passive, meaning that a chemical reaction in the sensor generates an electric current that can be sensed by a transmission element”.

The pH sensor is an electrochemical sensor. However, it is used to measure pH which can provide an indication of the health of the joint following implantation of an endoprosthesis, an indication of the presence of an infection in the joint before the infection is manifested by outwardly sensible symptoms (paragraph [0013]). Thus, it is used to indicate the presence of an infection and not to measure the implant's function and degradation during its life span.

The type of electrodes is different. In this document, “one such pH sensor that is suited for biomedical applications is an iridium oxide based potentiometric electrode sold by SenslrOx Inc” (paragraph [0049]).

EP 2 127 596: This document describes an implantable electrical sensor which is able to monitor conditions of the implant and of the tissue surrounding the implant. It can indicate if an implant is worn out and needs to be replaced (paragraph [0005]).

This sensor comprises an electrical circuit adapted to carry a current generated by a voltage difference applied to the electrical circuit. By determining the electrical resistance of the electrical circuit of the sensor, the rate of corrosion of the implant can be determined (paragraph [0017]). A current is generated by a voltage difference. This implies the presence of a voltage generator.

WO 2009/136167: This document relates to a system and a method for characterizing or monitoring the condition of implanted devices, such as a coronary stent, an endovascular stent, a percutaneous heart valve and replacement joints such as hip joints. The whole of implanted device surface is made of a conductive material. This arrangement permits the stent to be used as an electrode and also to be inductively coupled with a remote coil through which an electrical signal is passed. It is used for measuring the impedance and for determining a degree of restenosis associated with the device and the stage of tissue growth on the device.

The system disclosed is used to determine a degree of restenosis and not to measure the implant's function and degradation during its life span.

Other prior art publications are: WO 2009/095768, WO 02/064019, WO 01/49173, DE 102008005180, WO 2008/055229, WO 2006/089069, EP 1 677 225, WO 2005/120203, WO 2005/046467, US 2005/010299, US 2002/115944, WO 2008/022808, WO 01/35872, WO 2005/039440, US 5,935,171, US 2007/088442, US 2007/089518, US 2009/299228, US 2010/131067, US 2007/234819, WO 2005/084544, WO 2007/025191, WO 2008/035089, US 2006/232408, WO 01/18294, WO 97/33513.

### DESCRIPTION OF THE INVENTION

An aim of the present invention is to improve the known devices and methods.

More specifically, an aim of the present invention is to provide a system and method overcoming the defects of the prior art and allowing to monitor the in-vivo conditions of implants such as prosthesis.

A further aim of the present invention is to provide electrochemical sensors and techniques allowing the monitoring of the in-vivo conditions with high sensitivity thus providing a means to assess the surface condition of the implant.

Additionally, with the present invention, it is possible to obtain local information and thus identify the source of the problem.

A further aim of the present invention is use the system of the invention to induce an electrical current for stimulating bone growth around the stem of an implant or prosthesis in order to enhance post-operatively the implant anchoring in the bone of the patient being treated.

The present invention will be better understood by the detailed description of several embodiments thereof and from the drawings which show:

FIG. 1 illustrates a schematic representation of a hip joint replacement implant, the possible types of motion during use, and the resulting mechanisms of surface is degradation;

FIG. 2 illustrates a schematic representation of the implantable electronics system for measurement and communication according to the present invention;

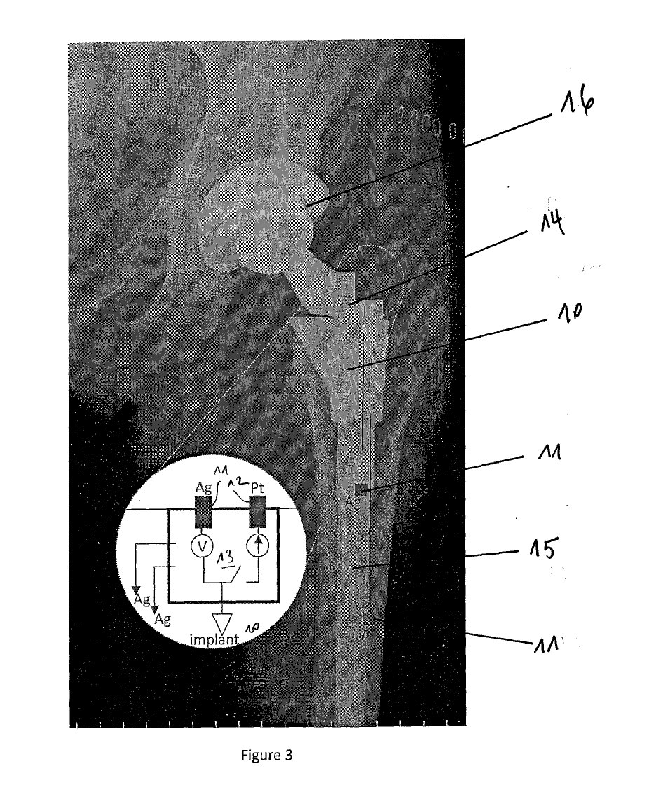

FIG. 3 illustrates a schematic diagram of a possible positioning of the electronic device and sensors on an intelligent prosthesis. Ag: Silver reference electrode. Pt: platinum counter electrode;

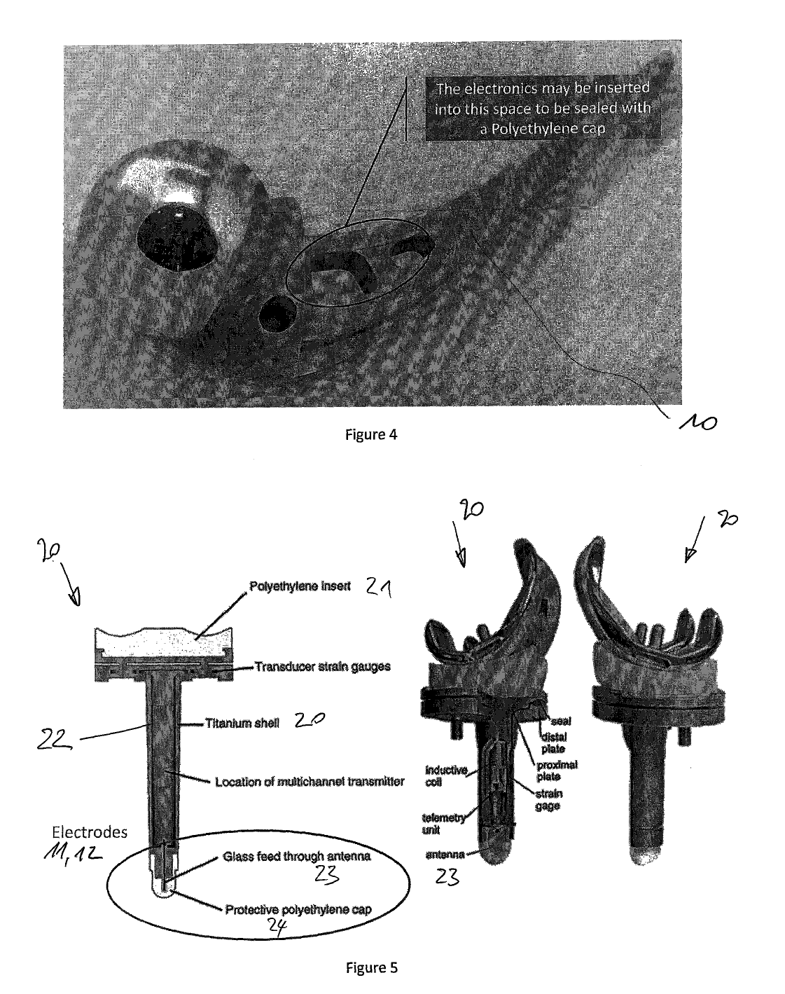

FIG. 4 illustrates possible position for integration of electronics in a hip replacement implant;

FIG. 5 illustrates possible position for integration of electronics in a knee replacement implant;

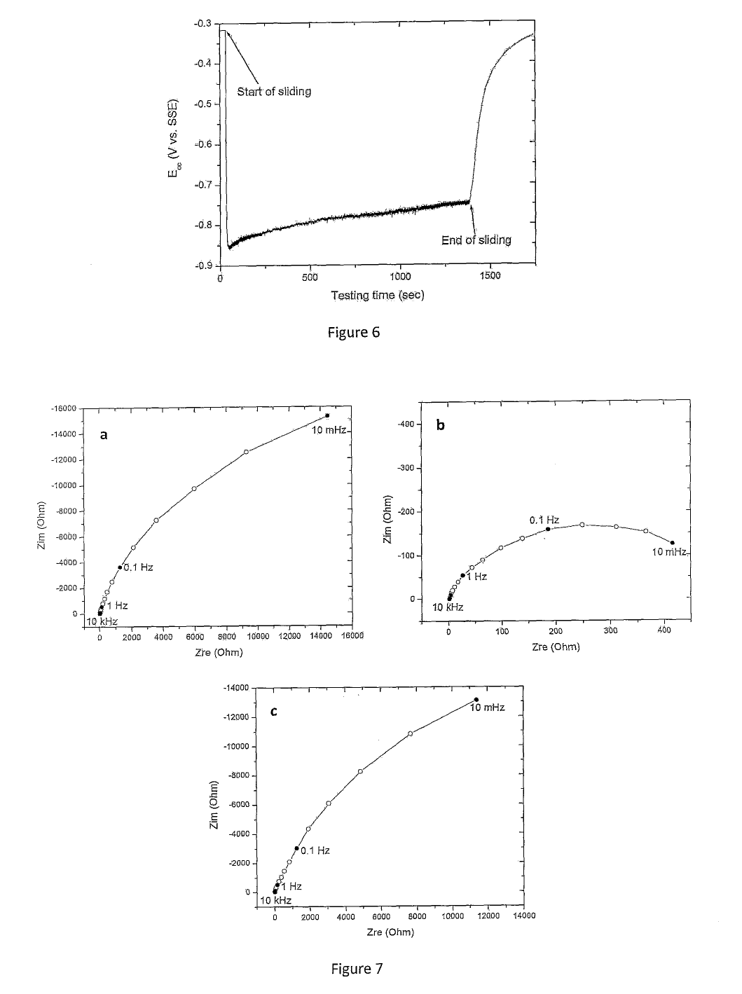

FIG. 6 illustrates the evolution of the open circuit potential before, during and after motion;

FIG. 7 illustrates the electrochemical impedance spectra before (a), during (b), and after (c) motion;

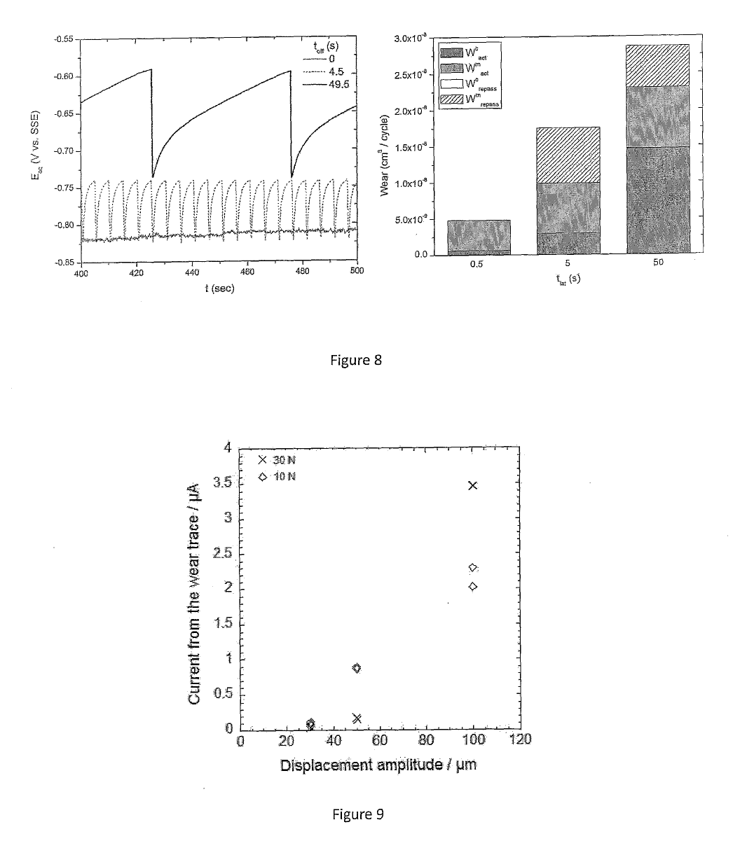

FIG. 8 illustrates the evolution of the open circuit potential during motion at different latency times (left), and the resulting evolution of the wear volume (right);

FIG. 9 illustrates the evolution of the current, i.e. the electrochemical wear, with the motion amplitude and the applied load during fretting;

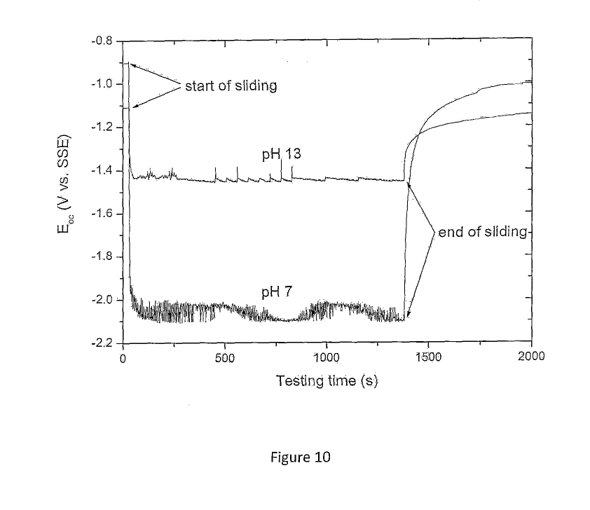

FIG. 10 illustrates the evolution of the open circuit potential before, during, and after motion in environments of different pH.

According to the present invention, an implantable electronic device is used to perform electrochemical tests and measurements on orthopedic or other implants after implantation and thereby allow an in-vivo monitoring of said implants.

Such devices have been designed to be miniature (few cm³) and have been shaped to be suitable for implantation (ultrathin) in the immediate neighborhood of e.g. a total hip joint replacement implant for example in this application.

Preferably, the electronic device is integrated in the implant or prosthesis to facilitate its use and application and take advantage of the implant's shape.

In one embodiment, the electronic device 3 according to the invention comprises measurement sensors 1, such as electrodes, signal processor 2, 6 means for the conditioning of the measured signal, a communication subsystem comprising a Radio Frequency (RFID) front-end 4 and its antenna 5 which allows a wireless external transfer and downloading of the measured data to an external control unit 9, a converter analogic-digital 6 and a control system 7. Preferably, the implanted device also comprise memory means 8 (RAM, ROM) able to store at least temporarily the acquired/measured data and other data such as communication protocols, and the data necessary for the functioning of the device.

Such a device may be constructed to be completely passive, i.e. without the use of an internal battery (which is in general the most cumbersome component) as an energy source and thus avoiding limitations in its life span. A schematical representation of the system architecture is shown in FIG. 2. In such case, the energy may be provided externally by the external control unit 9 or by a dedicated energy source as will be explained in more detail hereunder.

Use

In the current state we consider the following usage scenario. The measuring system as illustrated in FIG. 2 is implanted and connected to the main implant during surgery, for example hip joint replacement surgery in which an implant is placed in the body of a patient. The device according to the invention if integrated in the implant may thus not need a specific procedure but be implanted with the main implant/prosthesis.

Postoperatively the patient returns to the physician at a predetermined schedule for control testing. During testing, thanks to an external control unit 9 or reader, or a dedicated appropriate energy source, the physician may wirelessly energize and awaken the implanted measuring system for the test cycle. The measuring system performs the predetermined tests while the physician guides the patient to simulate different usage scenarios (standing, sitting, lying down, walking, running, climbing stairs etc.). At the end of the measuring cycle the data are wirelessly downloaded into the external reader 9 and are available for further processing for example by means of specific and dedicated programs and electronic means, such as a computer (not shown in the drawings but well known in the art).

Electrochemical Testing

Micro-electrodes 1 may be used as the measuring sensors in order to obtain information on the electrochemical properties of the joint replacement implant. In order for an electrochemical test to be applicable to living organisms, no significant current can be used. As a result, Open Circuit Potential (OCP), Electrochemical Impedance Spectroscopy (EIS), or Polarization Resistance (Rp) measurements as well as any other electro-analytical methods may be used in the frame of the present invention.

OCP measurements can be used to assess the degradation of the implant by e.g. comparing the data between an active (walking) and a passive (sitting or lying down) state of the patient.

An assessment of the degree of degradation due to motion can be achieved by OCP measurements, while pH changes may also be detected. OCP measurements are done using a 2-electrode setup (for example electrodes 11 and 12, see FIG. 3), where the potential of the metallic implant is measured against a known reference. The positioning of a number of reference electrodes at critical parts of the implant can provide local information on the surface surrounding each reference electrode.

EIS and Rp tests can provide more detailed information on the surface state of the implant and can lead to a quantification of the damage. Such tests are done using a 3-electrode setup (see the electrodes 1 in FIG. 2) where a potential is applied between the implant and the reference and the resulting current between the implant and the counter electrode is measured.

Construction

A schematic representation of an intelligent hip implant 10 according to the present invention with integrated electrochemical sensors 11, 12 and electronics 13 is shown in FIG. 3.

A critical parameter in the construction of the intelligent prosthesis according to the present invention is the positioning of the electronics 13 as well as the electrodes 11, 12. The electronics 13 need to be in a position where there is no contact with bones or other hard tissue while at the same time it does not impede the range of motion, mechanical integrity, or other functionality of the orthopedic implant. Furthermore, body fluids must not come in contact with the internal device components, and thus the electronics 13 container must be hermetically sealed.

However, the metallic parts of the prosthesis 10 hinder the propagation of the electromagnetic waves used for communications with the external reader 9. As a result, the RFID antenna 5 must be located outside a metallic part and sealed from the environment into a non-metallic casing that does not hinder wave propagation (e.g. polyethylene). A possible position close to the neck 14 of the stem of the hip implant 10 is shown in FIG. 3.

Other possible positions for the integration of the electronics into orthopedic implants are shown in FIGS. 4 and 5 for a hip 10 and a knee replacement prosthesis 20 respectively as illustrative embodiments.

In another embodiment, a second electronic device could be used at the acetabular cup which would provide information from the cup-bone interface. Electric connection to the implant is done at the position of the electronics.

As one will readily understand, the system of the present invention may be placed at different locations in order to monitor the in-vivo conditions at said locations.

A wide range of materials can be used for the reference electrodes 11. In FIG. 3, electrodes 11 made of Ag are shown as a reference against which the potential of the prosthesis is measured.

Ag is a candidate material for a reference electrode 11 since it is biocompatible and its potential depends mainly on the concentration of CI ions which is more or less stable in the human body.

Another possible material would be to use reference electrodes made of W. In that case the potential of the reference would depend on the pH of the environment. Other equivalent materials can also be considered in the frame of the present invention.

The reference electrodes 11 are positioned close to the areas of interest in order to obtain local information. Such areas are for example at the femoral head-acetabular cup interface 16, and along the stem 15 of the implant 10 inside the femoral bone (see FIG. 3). For the head-cup interface 16, a reference electrode positioned close or at the electronic device 13 may be used.

For the stem 15, a number of reference electrodes 11 may be positioned along its length and electrically connected to the electronic device 13 (two such electrodes are shown in FIG. 3). An additional electrode 12 made of Pt is also shown at the electronic device in FIG. 3. Such an electrode 12 is used as a counter electrode in order to perform tests where a small current is applied (see the EIS or Rp methods discussed above) and may be also used with this embodiment.

The electrical connection between electrodes 1, 11, 12 and electronics 3, 13 may be done in a number of ways:
From inside the prosthesis: hollow channels may be made to pass wire connections.
From the surface of the prosthesis: channels may be engraved but wires must be sealed from the environment.

In all cases, since no or only a very small current is passing during the electrochemical testing, only very thin wires are necessary and miniature feed-through connectors similar to the ones used in pacemakers may be used (e.g. Greatbatch).

FIG. 3 illustrates a Schematic diagram of a possible positioning of the electronic device and sensors on an intelligent prosthesis. Ag: Silver reference electrode 11. Pt: platinum counter electrode 12.

FIG. 4 illustrates a possible position for integration of electronics in a hip replacement implant. The electronics may be placed in lost space/volume of the prosthesis and sealed from the environment by an appropriate material.

FIGS. 5(A) to 5(C) illustrate a possible position for integration of electronics in a knee replacement implant. [D. D. D′Lima et al., J. Arthroplasty, 21 (2006) 255; B. Heinlein et al., Clin. Biomech. 24 (2009) 315].

Such implant 20 comprises a shell 21, for example made of titanium, and an insert 21, for example made of polyethylene.

The device according to the invention may be integrated in the stem 22 (see FIGS. 5(A) and 5(B), with an antenna 23 at the distal end of the stem 22 with a protective cap 24, for example made of polyethylene. The electrodes 11, 12, may be placed on the stem 22 according to the geometry disclosed in FIG. 3.

Supporting Results

Experimental results that illustrate the relationship between the use of a metallic implant and its electrochemical response are included in the present application. More specifically, the response of the open circuit potential at the beginning, during and after the end of motion (stand, walk, stand) is exhibited in FIG. 6 (Evolution of the open circuit potential before, during and after motion).

The EIS spectra obtained for a similar type of motion (stand, walk, stand) are shown in FIG. 7 (Electrochemical impedance spectra before (a), during (b), and after (c) motion.).

The evolution of the open circuit potential during motion at different latency times between each step (walking vs. running) and the resulting evolution of the wear volume are shown in FIG. 8 (Evolution of the open circuit potential during motion at different latency times (left), and the resulting evolution of the wear volume (right)).

The evolution of the anodic current which is a measure of the electrochemical wear of the implant during motion of different amplitudes (small steps vs. big steps), and at different applied loads is shown in FIG. 9 (Evolution of the current, i.e. the electrochemical wear, with the motion amplitude and the applied load during fretting).

The effect of the pH of the environment on the evolution of the open circuit potential before, during and after motion (stand, walk, stand) is shown in FIG. 10 (Evolution of the open circuit potential before, during, and after motion in environments of different pH).

The electrodes installed on prosthesis according to the principles exposed above may be used for the purpose of stimulating the growth of bone tissues in the area surrounding the implant as well.

This can be achieved by passing a small electrical current (typically 50 μA) between the implant and one or more electrodes 1, 11, 12 depending on the location and extent of the area were bone growth has to be promoted. In this way faster implant anchoring in the bone may be achieved after the surgery. Another benefit could be the repair of damaged bone around an implant without the needs of additional surgery.

Such mode of functioning may be carried out with the device according to the invention but functioning in another opposite mode as the in-vivo monitoring mode described above. Rather that reporting of certain modification in the electrochemical response (indicating a change in the in-vivo environment), the bone growth would be an active mode where some current is passed through the electrode for this purpose. The external reader 9 may be used for this purpose to transmit the necessary energy or another dedicated device.

As a consequence of this mode, the device according to the invention may be used in at least two modes, a first mode as an in-vivo monitoring device, and an active mode where bone growth is stimulated by the device.

The benefit of electrical currents to stimulate bone growth has been reported already at the end of the past century (Spadaro et al, Med. & Biol. Eng. & Comput., 1979, 17, 769-775 and A. A Marino (1988) Direct current and bone growth. In: Modern Bioelectricity (Edited by Marino A.), pp. 656-710, Marcel Dekker. New York.), but clinical products have not been yet marketed.

One of the probable reasons lies in the lack of wireless technology, which has been made available only in recent years. The need of external electrical contacts through hypodermic needles slid through the muscles and/or bone to reach the implant was a major problem especially considered the large time periods required for sufficient bone growth (typically 3 weeks of electrical treatment). The same device as developed for the in-vivo condition monitoring can be used for bone growth stimulation. For this the battery integrated in the control electronics inserted in the implant should be charged.

The electronics will afterwards apply a small current in the range 10-100 μA flowing between the implant 10 and the counter electrode 11 (or reference 12 electrode if material is suitable).

For this a small voltage of typically 1 V, corresponding to a power of 5 10-5 W is needed. The battery can be recharged at regular intervals during a treatment for example.

The present description provides exemplary embodiments and is not intended to limit the scope, applicability, or configuration of the claims. Rather, the present description will provide those skilled in the art with an enabling description for implementing the described embodiments. It being understood that various changes may be made in the function and arrangement of elements without departing from the spirit and scope of the appended claims.

For example, the material used for the electrode may be of any suitable type: as disclosed above in the embodiments of another equivalent material, i.e. biomedical, metals and alloys, for example the same materials as the one used for the implants etc.

In addition, the examples and values given above are only for illustrative purposes and should not be construed in a limiting manner. Different embodiments of the invention may be combined together according to circumstances. In addition, other embodiments and applications may be envisaged for example by using equivalent means.

1. A metallic prosthesis to be implanted in a body, for example a human or animal body, which contains means to monitor its condition during use in order to allow an early detection of failure or insufficient functionality, and/or to apply a stimulating condition, wherein said means comprise at least implanted sensors with electrodes and electronic device and a remote device, to measure the implant's function and degradation during its life span, wherein said sensors are electrochemical sensors.

2. The prosthesis according to claim 1, wherein said electronic device comprises at least a signal processor, a communication subsystem.

3. The prosthesis according to claim 1, wherein the communication subsystem comprises a Radio Frequency front-end and its antenna which allows the wireless external downloading of the measured data by the electrodes or the application of energy to apply the stimulating condition.

4. The prosthesis as defined in claim 3, wherein the electronic device is passive and said remote device comprises an energy source.

5. The prosthesis according to claim 1, wherein micro-electrodes are used.

6. The prosthesis according to claim 1, wherein the electrodes are made of Ag or tungsten, or another equivalent material.

7. The prosthesis according to claim 1, wherein said prosthesis is a hip prosthesis.

8. The prosthesis according to claim 1, wherein said prosthesis is a knee prosthesis.

9. A method of measuring the functionality of an implanted prosthesis using the prosthesis as defined in claim 1.

10. The method according to claim 9, wherein measuring the functionality of an implanted prosthesis utilizes one or more of Open Circuit Potential (OCP), Electrochemical Impedance Spectroscopy (EIS), or Polarization Resistance (Rp) measurements.

11. A method of using an implanted prosthesis with electrodes as defined in claim 1, wherein said prosthesis is used to promote bone growth by applying an electric current to the environment of the electrodes of said prosthesis.
